(19) United States
(12) Patent Application Publication
(10) Pub. No.: US 2015/0278901 A1
(43) Pub. Date: Oct. 1, 2015

(54) AUTOMATED PRODUCT CUSTOMIZATION BASED UPON LITERATURE SEARCH RESULTS

(71) Applicant: FIREFLY BIOWORKS, INC., Cambridge, MA (US)

(72) Inventors: Andreas Windemuth, Belmont, MA (US); Isaac Stoner, Cambridge, MA (US); Davide Marini, Cambridge, MA (US); Daniel Pregibon, Somerville, MA (US)

(21) Appl. No.: 14/439,530

(22) PCT Filed: Nov. 5, 2013
(86) PCT No.: PCT/US13/68584

§ 371 Date: Apr. 29, 2015

Related U.S. Application Data
(60) Provisional application No. 61/722,672, filed on Nov. 5, 2012.

(51) Int. Cl. G06Q 30/06; G06F 19/16; G06F 19/18; G06F 17/30

(52) U.S. Cl. CPC G06Q 30/0621; G06F 17/30867; G06F 19/16; G06F 19/18

(57) ABSTRACT

In certain embodiments, the invention relates to systems, methods, and apparatus that allow a purchaser to customize a test kit based upon a literature search. A processor receives one or more search terms entered by a user and constructs a query comprising the one or more search terms. The processor obtains from one or more literature repositories a plurality of search results responsive to the query. The processor identifies one or more domain terms (relating to a scientific domain) within the plurality of search results. The processor prioritizes the one or more scientific domain terms based in part upon frequency of occurrence within the plurality of search results, and selects one or more order components relating to a tangible product, each order component relating to a respective scientific domain term of the one or more scientific domain terms. The order information is displayed to a user.

### RELATED APPLICATIONS

The present application claims priority to and the benefit of, U.S. Provisional Patent Application No. 61/722,672, filed Nov. 5, 2012, titled “Automated Product Customization Based Upon Literature Search Results,” the contents of which is incorporated herein by reference in its entirety.

### TECHNICAL FIELD

The present invention relates generally to automated customization of a tangible product, for example, a biological assay.

### SUMMARY

Described herein are various embodiments of systems, methods, and apparatus that allow a purchaser to customize a test kit based upon a literature search. In selecting a test kit, such as a kit including a series of test components for testing a biological sample, a purchaser may desire help in identifying one or more relevant components. The components may be relevant, for example, based upon one or more goals of the purchaser. For example, if a purchaser is interested in learning about a particular biological pathway, the purchaser may want to learn more about which microRNA biomarkers have been associated with the biological pathway in recent studies. An automated literature search may be used during the purchase process, in this example, to swiftly identify microRNA biomarkers related to the biological pathway based upon an association between each identified microRNA biomarkers and the biological pathway within a body of literature. The body of literature, in some examples, may include online journals, scientific research repositories, university digital libraries, and other available databases containing scientific research collections. The body of literature, in some implementations, may be queried based in part on the relevance in a particular area of research. For example, one or more first literature repositories may be searched in relation to microRNA biomarkers, while one or more second literature repositories may be searched in relation to proteins. There may be some overlap between the one or more first literature repositories and the one or more second literature repositories.

In various embodiments, the systems, methods, and apparatus utilize or include a tablet computer, a mobile phone device, or any other computer device or system capable of receiving input. A web site interface, mobile device application, customized computer application, or other electronic order system is used to connect the user (e.g., at the tablet computer, mobile phone device, or other computer device) with an ordering system including a query mechanism for researching one or more literature sources to identify relevant items to include within the product customization. The systems, methods, and apparatus have applications in a wide variety of industries that supply scientific research products, testing systems, or testing services.

Elements of embodiments described with respect to a given aspect of the invention may be used in various embodiments of another aspect of the invention. For example, it is contemplated that features of dependent claims depending from one independent claim can be used in apparatus, articles, systems, and/or methods of any of the other independent claims.

One aspect presented herein relates to a method including receiving, via a network, one or more user search terms entered by a user at a remote computing device; constructing, by a processor of a computer device, a query, wherein the query includes the one or more user search terms and/or is determined based at least in part on the one or more user search terms; obtaining, from one or more third party literature repositories, a plurality of search results responsive to the query; identifying, by the processor, one or more scientific domain terms within the plurality of search results, wherein each of the one or more scientific domain terms relates to one or more of a set of predetermined scientific domain categories; prioritizing, by the processor, the one or more scientific domain terms based at least in part upon frequency of occurrence within the plurality of search results; selecting, by the processor, one or more order components relating to a tangible product, wherein each order component of the one or more order components relates to a respective scientific domain term of the one or more scientific domain terms; and providing, for display to the user at the remote computing device, order information including the one or more order components.

Another aspect presented herein relates to a system including a processor; and a memory having instructions stored thereon, wherein the instructions, when executed by the processor, cause the processor to: receive, via a network, one or more user search terms entered by a user at a remote computing device; construct a query, wherein the query includes the one or more user search terms and/or is determined based at least in part on the one or more user search terms; obtain, from one or more third party literature repositories, a plurality of search results responsive to the query; identify one or more scientific domain terms within the plurality of search results, wherein each of the one or more scientific domain terms relates to one or more of a set of predetermined scientific domain categories; prioritize the one or more scientific domain terms based at least in part upon frequency of occurrence within the plurality of search results; select one or more order components relating to a tangible product, wherein each order component of the one or more order components relates to a respective scientific domain term of the one or more scientific domain terms; and provide, for display to the user at the remote computing device, order information including the one or more order components.

An additional aspect presented herein relates to a non-transitory computer readable medium having instructions stored thereon, wherein the instructions, when executed, cause the processor to: receive, via a network, one or more user search terms entered by a user at a remote computing device; construct a query, wherein the query includes the one or more user search terms and/or is determined based at least in part on the one or more user search terms; obtain, from one or more third party literature repositories, a plurality of search results responsive to the query; identify one or more scientific domain terms within the plurality of search results, wherein each of the one or more scientific domain terms relates to one or more of a set of predetermined scientific domain categories; prioritize the one or more scientific domain terms based at least in part upon frequency of occurrence within the plurality of search results; select one or more order components relating to a tangible product, wherein each order component of the one or more order components relates to a respective scientific domain term of the one or more scientific domain terms; and provide, for display to the user at the remote computing device, order information including the one or more order components.

In some embodiments, the set of predetermined scientific domain categories includes one or more of molecular biology, proteins, immunoassays, genetic polymorphisms, genomic assays, RNA molecules, expression analysis, messenger RNA, small RNA molecules, microRNA molecules, and/or any combination thereof. In some embodiments, a first repository of the one or more third party literature repositories is PubMed. In some embodiments, the query includes a domain term. In some embodiments, constructing the query includes identifying the domain term relevant to the user. In some embodiments, identifying the domain term includes identifying the scientific domain based upon the one or more terms entered by the user. In some embodiments, identifying the one or more domain terms includes using a pattern matching algorithm to identify patterns within text of the plurality of search results. In some embodiments, each search result of the plurality of search results includes at least one of a summary, an abstract, an article, and a publication.

In some embodiments, third party literature repositories are copied in whole or in part to be stored locally on a server associated with the processor, to improve response time.

In some embodiments, the method further includes additionally filtering the one or more domain terms to remove one or more least frequently identified domain terms. In some embodiments, the method further includes, or the instructions stored on the computer medium or the memory cause the processor to, additionally filter the one or more domain terms to remove one or more least frequently identified domain terms. In some embodiments, the method also includes filtering the plurality of search results to remove duplicates. In some embodiments, the instructions, when executed, cause the processor to additionally filter the plurality of search results to remove duplicates.

In some embodiments, prioritizing the one or more scientific domain terms includes scoring the one or more scientific domain terms by at least the frequency of occurrence. In some embodiments, the method further includes, prior to selecting the one or more order components, providing, for display to the user at the remote computing device, the one or more domain terms, wherein providing the one or more domain terms includes providing a respective score associated with each domain term of the one or more domain terms. In some embodiments, the instructions stored on the computer medium or the memory cause the processor to, prior to selecting the one or more order components, provide, for display to the user at the remote computing device, the one or more domain terms, wherein providing the one or more domain terms includes providing a respective score associated with each domain term of the one or more domain terms. In some embodiments, the method further includes, additionally receiving, via the network, responsive to providing the one or more domain terms, at least one of an addition of a domain term and a removal of a domain term of the one or more domain terms. In some embodiments. the instructions stored on the computer medium or the memory cause the processor to, additionally receive, via the network, responsive to providing the one or more domain terms, at least one of an addition of a domain term and a removal of a domain term of the one or more domain terms. In some embodiments, the method further includes, prior to selecting the one or more order components, receiving an indication from the user to initiate generation of a product order based upon the one or more domain terms. In some embodiments, the instructions stored on the computer medium or the memory cause the processor to, prior to selecting the one or more order components, receive an indication from the user to initiate generation of a product order based upon the one or more domain terms.

In some embodiments, the tangible product includes a biological panel for detection of a plurality of identified microRNA targets, a multiplex biological panel, a therapeutic or combination of therapeutics, a PCR primer set, and/or one or more small RNA mimics. In some embodiments, the multiplex biological panel includes a customized multiplex panel for detection of one or more identified proteins, messenger RNAs, SNPs, and/or genetic variations thereof. In some embodiments, the one or more small RNA mimics include(s) microRNAs, lncRNAs, siRNAs, anti-microRNAs, piwiRNAs, and/or any combination thereof.

Another aspect presented herein relates to a method for automated (or semi-automated) selection or customization of a tangible product. The method includes providing a first graphical user interface for display on a user computing device, said interface being configured to accept user input from the user computing device; receiving, via a network, a first set of user input from the user computing device, said first set of user input including a first set of one or more tokens and, optionally, one or more of the following: (i) a selected organism, and (ii) a selected scientific domain. The method also includes accessing, by the processor, one or more databases and performing, by the processor, a first query of the one or more databases using said first set of user input to identify a set of initial search results; transmitting, for graphical display on the user computing device, the set of initial search results for rendering on a display of the user computing device; providing, for graphical display on the user computing device, a graphical user interface (GUI) widget (e.g., a control button) that, upon selection by the user, initiates preparation of an order form providing specifications for a customized tangible product corresponding to at least a subset of the initial (or subsequent) search results; providing, for graphical display on the user computing device, the order form, said order form including a listing of selectable attributes corresponding to said specifications of the tangible product.

In some embodiments, the method additionally includes providing, for graphical display on the user computing device, a link corresponding to each of one or more members of the set of initial search results rendered on the display of the user computing device (e.g., including the order form), said link(s) including an identification of a microRNA (or messenger RNA, SNP, or genetic variation) search result which, upon selection of the link by the user, presents a listing of documents from the one or more databases describing the selected microRNA (or messenger RNA, SNP, or genetic variation) and, optionally, presents links to said documents.

In some embodiments, the method also includes receiving, via the network, a second (or subsequent) set of user input from the user computing device following display of the set of initial (or prior) search results on the display of the user computing device, said second (or subsequent) set of user input including a second (or subsequent) set of one or more tokens (e.g., alphanumeric user search term (word, phrase), image, graphical chemical structure, or graphical biological structure), and performing, by the processor, a second (or subsequent) query of the one or more databases using said second (or subsequent) set of user input to identify a set of subsequent search results. In some embodiments, the second (or subsequent) set of user input is a selection by the user of one or more search results previously presented to the user (e.g., a term presented in the plurality of graphical cloud representations). In some embodiments, the method also includes providing, for graphical display on the user computing device, a GUI widget that, upon selection by the user, instructs merging, by the processor, of the first query with the second query of the one or more databases to identify the set of subsequent search results.

In some embodiments, the method also includes providing, for graphical display on the user computing device, a plurality of graphical cloud representations, each graphical cloud representation configured to convey a visualization of a score corresponding to each of the set of initial search results, wherein the plurality of graphical cloud representations include one or more of the following: (i) a key word cloud including words found in texts identified in the first query; (ii) a microRNA cloud including designations of microRNAs in texts identified in the first query; (iii) an author cloud including designations of authors of texts identified in the first query; and (iv) a gene cloud including designations of genes in texts identified in the first query.

In some embodiments, the method also includes transmitting the completed order form to a manufacturer for fulfillment of the tangible product. In some embodiments, the tangible product is a new, customized product. In some embodiments, the tangible product is an existing product. In some embodiments, the tangible product includes a biological panel for detection of a plurality of identified microRNA targets; a multiplex biological panel; a therapeutic or combination of therapeutics; a PCR primer set; or one or more small RNA mimics.

In some embodiments, the multiplex biological panel includes a customized multiplex panel for detection of one or more identified proteins, messenger RNAs, SNPs, and/or genetic variations thereof. In some embodiments, the one or small RNA mimics includes microRNAs, lncRNAs, siRNAs, anti-microRNAs, piwiRNAs, and any combination thereof. In some embodiments, the first set of one or more tokens includes an alphanumeric user search term (word, phrase), image, graphical chemical structure, or graphical biological structure, or any combination thereof. In some embodiments, the selected organism is a human or a mammal. In some embodiments, the selected scientific domain includes microRNAs, proteins, genes, and/or any combination thereof. In some embodiments, the one or more databases includes biological databases, medical databases, and/or scientific literature databases. In some embodiments, the one or more databases is PubMed, NIH Gene Expression Omnibus (GEO) datasets, CMAP/Connectivity Map datasets, and KEGG database (Kyoto Encyclopedia of Genes and Genomes).

In some embodiments, the set of initial search results is rendered on the display of the user computing device as an initial search results page including a plurality of graphical cloud representations, each graphical cloud representation including (or being configured to convey) a visualization of a score corresponding to each of the set of initial search results. In some embodiments, the score is a function of frequency of occurrence within the searched database(s). In some embodiments, the score is represented graphically by font size and/or color. In some embodiments, only search results exceeding a given score are graphically displayed.

In some embodiments, the selectable attributes include specific microRNA targets, specific messenger RNAs, specific SNPs, specific genetic variations. In some embodiments, the selectable attributes include specific therapeutics, elements of a PCR primer set, or small RNA mimics. In some embodiments, wherein each of the selectable attributes includes a GUI widget displayed on the order form allowing selection or deselection of said attribute by the user, said selected attributes (following selection by the user of a subset of said displayed selectable attributes) corresponding to the specifications for the customized tangible product.

In some embodiments, the method also includes identifying, by the processor, one or more scientific domain terms within the set of initial search results, wherein each of the one or more scientific domain terms relates to one or more of a set of predetermined scientific domain categories. In some embodiments, the method also includes prioritizing, by the processor, the one or more scientific domain terms based at least in part upon frequency of occurrence within the set of initial search results.

In some embodiments, the one or more databases is a third party literature repository. In some embodiments, at least a portion of the one or more databases is downloaded and/or stored on a server associated with the processor. In some embodiments, the one or more databases is stored locally on a server associated with the processor.

### BRIEF DESCRIPTION OF THE DRAWINGS

The foregoing and other objects, aspects, features, and advantages of the invention will become more apparent and may be better understood by referring to the following description taken in conjunction with the accompanying drawings, in which:

FIG. 1 is a block diagram of an example system for automating product customization based upon literature search results;

FIG. 2 is a flow chart of an example process for automating product customization based upon literature search results;

FIGS. 3A through 3C are example screen shots of a user interface for ordering a product that has been automatically customized based upon literature search results;

FIGS. 4A through 4G are example screen shots of a user interface for ordering a product that has been automatically customized based upon literature search results;

FIG. 5 is a block diagram of another example network environment for creating software applications for computing devices;

FIG. 6 is a block diagram of a computing device and a mobile computing device.

The features and advantages of the present disclosure will become more apparent from the detailed description set forth below when taken in conjunction with the drawings, in which like reference characters identify corresponding elements throughout. In the drawings, like reference numbers generally indicate identical, functionally similar, and/or structurally similar elements.

### DESCRIPTION

It is contemplated that apparatus, systems, and methods of the claimed invention encompass variations and adaptations developed using information from the embodiments described herein. Adaptation and/or modification of the apparatus, systems, and methods described herein may be performed by those of ordinary skill in the relevant art.

It should be understood that the order of steps or order for performing certain actions is immaterial so long as the invention remains operable. Moreover, two or more steps or actions may be conducted simultaneously.

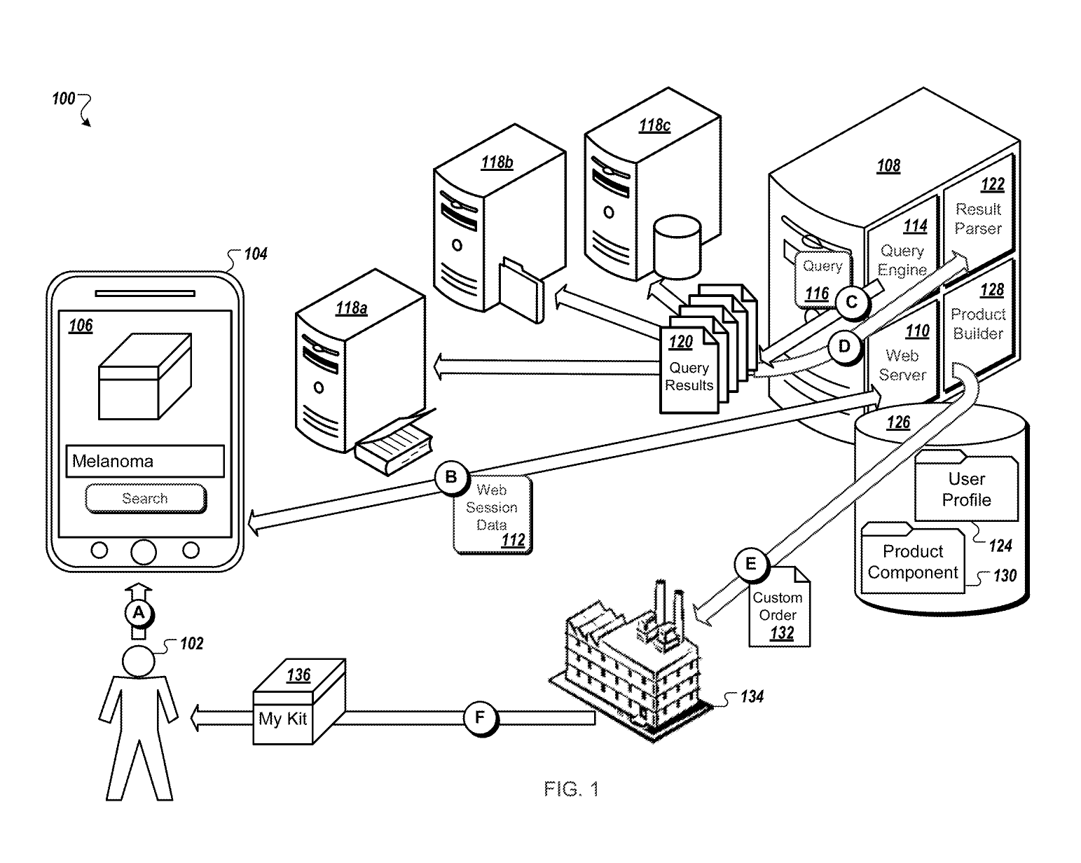

FIG. 1 is a block diagram of an example system 100 for automating product customization based upon literature search results. The system, for example, is configured to provide a user 102 at a computing device 104 with a graphical interface 106 for exploring product order customizations through a third party literature search.

The system 100 includes a server 108 configured with a web server 110 to share interactive session data 112 with the computing device 104. The user at the computing device 104, for example, may provide one or more tokens (e.g., word, phrase, image, graphical chemical structure, multiple choice selection, etc.) to the server 108 as session data 112. A query engine 114 of the server 108 may construct a query 116 based in part upon the token(s) provided by the user 102. The query 116, in some implementations, includes a domain, such as a category, scientific field, or other classification to narrow the results in relation to the token(s) provided by the user 102. In some implementations, the domain is based in part upon a web page accessed by the user 102. For example, if the graphical interface 106 presented on the computing device 104 has been presented in relation to ordering a microRNA testing kit, the domain may include the term “microRNA”.

The query engine 114, in some implementations, presents the query to one or more third party literature sources 118 such as, in some examples, a publisher server 118a, a file repository server 118b, and a database server 118c. One or more of the third party servers 118, in some implementations, are specialized servers for accessing scientific literature. The scientific literature, for example, may include university theses, laboratory studies, journal articles, medical industry standards, peer-reviewed experiment results, and other reliable scientific data. In some implementations, a particular third party server 118 or servers 118a are identified by the query engine 114 based in part upon the domain of the query 116. The domain of the query 116, in some implementations, may be used to query a particular collection provided by a particular third party server 118.

In response to the query 116, the server 108 receives query results 120. The query results 120, in some examples, may include abstracts, summaries, articles, and synopses related to the query 116. The query results 120 are provided to a result parser 122 of the server 108 to parse relevant information from the query results 120. The result parser 122, for example, may identify domain terms related to the domain of the query 116 within the query results 120. In some implementations, the result parser 122 identifies microRNA biomarkers in relation to a query performed in a microRNA domain. The result parser 122, in some implementations, removes duplicate results from the query results 120, sums the number of results related to each of the domain terms, and ranks the identified domain terms. Ranking of the identified domain terms, for example, may be based in part on one or more of recency, frequency, type of query result (e.g., article vs. press release, etc.), relative reliability of the source (e.g., peer-reviewed journal article versus university thesis, etc.), and popularity of the domain term (e.g., based upon historic similar orders as identified within user profile data 124 of a data store 126).

Web session data 112, in some implementations, is provided to the user 102, allowing the user 102 to review the domain terms and/or query results 120. For example, the user 102 may be provided the opportunity to remove domain terms, add a domain term, or discard a portion of the query results 120.

In some implementations, based upon the identified domain terms, a product builder 128 of the server 108 prepares a customized product order including product components related to the identified domain terms. The product builder 128, in some implementations, associates the domain terms with the product components by accessing product component data 130. The product builder 128, in some implementations, presents order information to the user 102 via web session data 112.

Upon approval of the order information by the user 102, in some implementations, the server 108 provides customized order specifications 132 for preparation of an order (e.g., at an order facility 134). Upon preparation, an order 136, in some implementations, is shipped to the user 102.

Examples below illustrate a variety of uses for the system 100 to customize various products.

### Example N.1

### Life Sciences

Without limiting the applicability of the present invention to the field of biotechnology, it is helpful to describe one of its possible embodiments in this field. Life sciences researchers are often in need of profiling their samples for the presence and amount of a variety of biologically important molecules, such as proteins, nucleic acids and other biomarkers. These tests are most effectively performed when all the biomarkers of interest can be measured contemporaneously in the same sample, a technique known in the art as “multiplexed detection.” Due to the extraordinary complexity of biological systems and the vast amount of biomarkers that could be of importance to a specific experiment, scientists need to choose what to measure and what to neglect. In order to make such selection, researchers typically rely on their own experience and insight, as well as on searching the literature for results published by other scientists working on a similar question or biological pathway. This process can often be tedious and lends itself to non-objective and non-data-driven decisions.

Here we describe a method for creating and purchasing a product specifically customized to a scientist's needs, by automatically searching the scientific literature for instances of published data containing biomarkers relevant to a specific inquiry, ranking them according to citation frequency, building and purchasing a custom product to measure all the biomarkers specified by the search algorithm.

A specific embodiment of this invention can be exemplified in the area of microRNA research. microRNAs are short, non-coding RNAs that regulate gene expression and have recently shown great potential as cancer biomarkers. There are about one thousand microRNAs in the human genome and each is devoted to regulating a specific set of genes. A clinical scientist working in the field of melanoma, for example, may wish to profile clinical samples for the presence of specific microRNAs that may be indicative of cancer propensity in the tissue to be analyzed. Given the rapid progress of the field, the scientist may wish to know what set of microRNAs could me most relevant to melanoma, in particular as a result of data already published in the literature. Rather than manually searching all the published papers, selecting the most relevant microRNAs and ordering a corresponding assay, our system streamlines the whole process, allowing the scientist to simply type the word “melanoma” in a search interface and have a computer search the published literature, rank the data appropriately and objectively, interface with the production line of a company, and have the customized product ship directly to the scientist's laboratory.

### Example N.2

### Personalized Meals

The need exists for individuals with metabolic conditions to design a meal plan that takes into account, for example, the glycemic index of various foods, as well as the most recent data from clinical studies related to nutrition. As an example of an embodiment of the current invention, a person would type in a search engine the words “diabetes, pumpkin, spinach, pasta”. The system automatically searches the published literature for the glycemic impact of those foods, designs a pasta formulation that, while satisfying the requirement for the presence of all those ingredients, mixes them in the optimal proportion for minimizing glycemic impact. The system then interfaces with the production lines of a pasta manufacturer and sends a specific order to the assembly line, packages the pasta in boxes and ships the finished product to the customer.

### Example N.3

### Well-Visit Test Panels

Patients require vastly different panels of blood workups and monitoring tests at regularly scheduled checkups and wellness visits. Current standardized testing criteria for tests fail to account for myriad combinations of conditions and risk factors. These could include gender, race, ethnicity, height, weight, age, family history, genotype/sequence data, or pre-existing conditions or disease states. Clinicians require an automated method in order to select from the ever-increasing number of sophisticated diagnostic/prognostic methods becoming available. An embodiment of this invention could include an algorithm to automatically search databases of biomarker tests (diagnostic, prognostic, or other) based on specific patient data. The most medically relevant tests would be returned to the clinician, and could be further filtered based on best practices, regulatory approval, reimbursement status, cost, or test sensitivity/specificity data. Upon finalizing the selection, the system will incorporate all of the tests into a unified purchase order, so the patient can order all the tests to be performed.

### Example N.4

### Guiding Clinicians to Genome Locations/Sequence Variants of Interest

Genetic analysis techniques such as sequencing have become clinically relevant and will continue to be important in molecular diagnostics and other areas of medicine. As these techniques become exponentially faster and cheaper, clinicians will soon be faced with a glut of data that will prove extremely challenging to analyze. Tools that allow clinicians to hone in on gene variants or other regions of the genome that may be involved in specific diseases will prove invaluable. One embodiment of this invention could include an algorithm to automatically parse public databases of sequencing data or bodies of literature associating certain genotypes/sequences with diseases based on clinical indicators. A user would input clinical indications, which could include diseases, symptoms, risk factors, lab test results, biomarkers, or some combination. The algorithm would output suggestions for genome locations, variants, genotypes, single-nucleotide polymorphisms or some combination of these that are likely to be associated with the clinical indications entered by the user.

### Example N.5

### Therapeutics

As more research is performed, the connection between diseases and therapeutic agents becomes clearer through understanding the roles of genes, proteins, and non-coding RNAs in disease onset, progression and treatment. These biomarkers may be related to diseases either directly, or indirectly through the involvement of other biomarkers. A search engine is provided in which a user enters a disease or set of symptoms into a search field, and the engine returns a list of therapeutic agents that could be used, singly or in combination, for treatment. This can be based, for example, on the most up-to-date academic publications or clinical trial results. This can be based on complex networks of association discovered through literature searches across several molecule classes. For example, if a set of scientific articles may describe how disease D is caused by a defect in protein P, a second set of articles describes how the translation of the mRNA for protein P is regulated by microRNA M, and a third set of articles describes how the function of M can be silenced using therapeutic T, then through association, the search algorithm can indicate that T can potentially be used to treat D. This process would require querying of one or more databases, parsing through titles and abstracts to find leads, scoring of potential associations, and display of results in a manner that is thorough, but manageable.

### Example N.6

### PCR Primer Design for Targeted Enrichment of Genetic Sequences

To date, genome-wide association studies have failed to deliver on the promise of next-gen sequencing (NGS). These genome-wide approaches often give clinicians more data than is clinically actionable, and may raise ethical issues around patient privacy and right-to-know. More selective targeted sequencing approaches will become more important as NGS enters the clinic. As the demand for these PCR-based targeted enrichment approaches increases, medical personnel and clinical researchers will need an automated way to design and order PCR primers to amplify gene regions of interest. In one embodiment, a user would enter the disease, transcript, or genome region of interest and an algorithm would search scientific databases or sequence repositories of interest to automatically design primer pairs. The algorithm could further optimize these primer pairs for multiplexed PCR reactions, minimizing non-specific interactions such as primer-dimer formation. Finally, the algorithm would facilitate purchase of these primer sets directly from the search results.

### Example N.7

### Small-RNA Mimics for Therapy

A new paradigm in drug design has begun, utilizing the delivery of small-RNA mimics, such as microRNA mimics, in order to selectively inhibit or enhance given biological pathways. As delivery methods become increasingly effective, these non-toxic biomolecules will become omnipresent as therapeutic agents. An automated method for selecting small RNAs (such as microRNAs, lncRNAs, siRNAs, anti-microRNAs, piwiRNAs, etc.) will allow for creating designer treatments based on the delivery of one or a combination of these biomolecules. One embodiment would allow clinical researchers or designers of therapeutics to enter a condition or morbidity. The algorithm would then automatically parse databases of scientific literature, small RNAs, and disease pathways in order to select for the most likely small RNA transcripts of interest. The algorithm would then allow for users to directly order one or a combination of these small-RNA mimics, or inhibitory reverse complements to these molecules, for therapeutic investigation or applications.

Other embodiments are possible while staying within the scope and purpose of the system 100. Various embodiments and features are further described below.

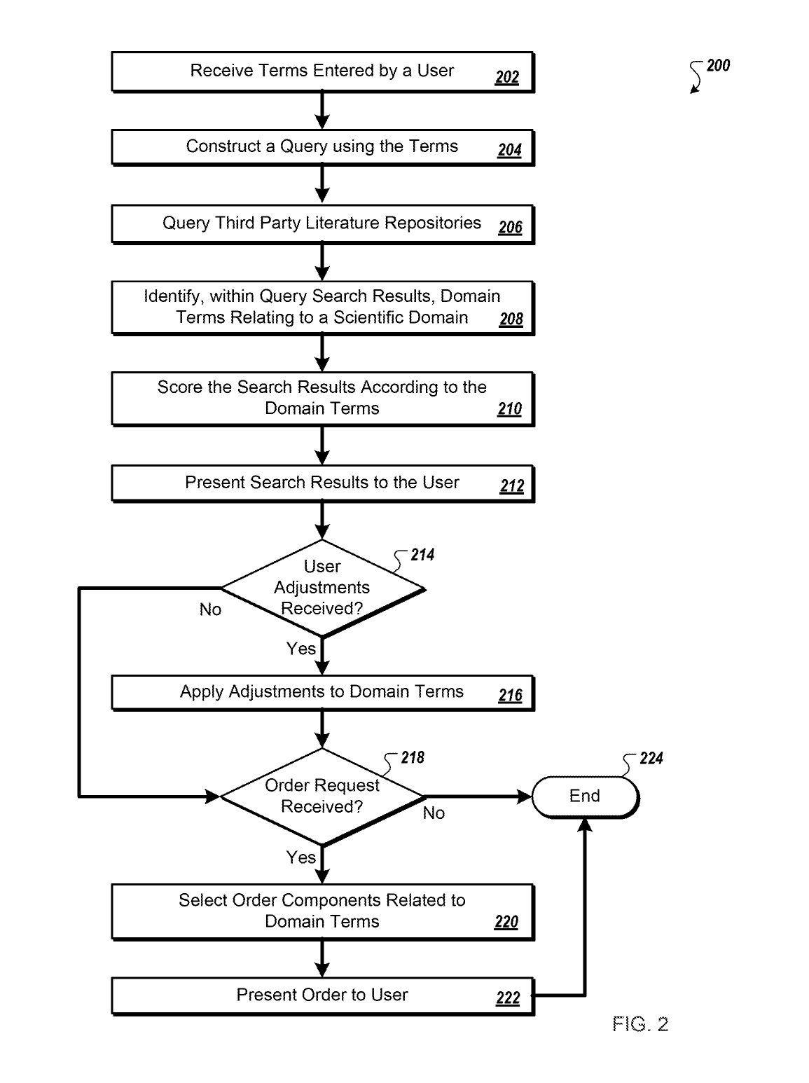

FIG. 2 is a flow chart of an example method 200 for automating product customization based upon literature search results.

In some implementations, terms entered by a user are received (202).

In some implementations, a query is constructed using the terms (204). When a search term is entered by the user, in some implementations it is first analyzed using a domain recognition algorithm. If a query term identifies a domain (e.g., “microRNA”, “DNA”, etc.), in some implementations it remains unchanged. If the terms provided by the user do not include a domain, in some implementations, text identifying a domain, such as the text “AND microRNA,” is appended to restrict the search to results relevant to microRNA research. In other examples, the domain may identify a term or phrase relevant to biomolecule detection, the detection of proteins, messenger RNAs, single-nucleotide polymorphisms, or genetic variations. Searches may be designed across multiple target classes, in some implementations, to include various combinations of biomolecules in a single product.

In some implementations, one or more third party repositories are queried (206). The resulting query, for example, is then sent to a remote search engine, such as PubMed. In some implementations, the query includes instructions relevant to a particular third party query server. For example, a particular query server may accept instructions on results formatting, such as instructions to return title and abstract plus other relevant information for up to 200 of the top results of the search. If multiple third party repositories are queried, in some implementations, equivalent instructions may be provided to each repository. In some implementations, repositories may be copied in whole or in part to be stored locally on the server, to improve response time.

In some implementations, domain terms are identified within the search results responsive to the query (208). For example, the search results may be scanned (e.g., as arbitrary text) for the occurrence of microRNA designations. This may be done, for example, using a pattern match with the well-known regular expression algorithm. In a particular example, the pattern that is scanned for is either of the terms “microRNA” or “miR”, followed by a number, a letter, and possibly another number which together constitute the full identifier for a microRNA species. The algorithm, in some implementations, contains multiple provisions for variations in the text, such as upper case vs. lower case, presence or absence of hyphens, etc. In some implementations, text in the search results may be matched against a dictionary of domain terms. For example, in some implementations, text may be matched against the GenBank database of gene names to identify genes mentioned in the text. Multiple domains may be parsed concurrently, with ambiguities resolved by considering the frequency of use and/or other property or combination of properties of each dictionary entry from different domains. The dictionary may contain the official entity names or identifiers, as well as any aliases that may be in use to refer to them in the literature.

In some implementations, the search results are scored according to the domain terms (210). In some implementations, the text of the query results is analyzed to score all domain terms, for example based upon one or more of frequency journal impact factor, article author, date of publication, and product order patterns. In some embodiments, filters and scoring metrics may be customizable or user-defined. In some implementations, the score of each distinct domain term is used to generate a visualization plot for presentation to the user.

In some implementations, search results are presented to the user (212). For example, the visualization plot related to the domain term scoring may be presented to the user. In some implementations, a reference record is created for each mention of a particular domain term. The reference record, for example, may link each domain term to one or more query results in which the domain term is mentioned. The reference records, for example, may be used in presenting search results to the user.

In some implementations, the query results are parsed to obtain particular relevant information such as, in some examples, title, abstract, authors, year and journal information for the top results. The particular relevant information, in some implementations, is further broken down for term extraction. For example, all titles and abstracts may be broken down into individual words and filtered to exclude common English words. In a particular embodiment, after filtering, the remaining words are given a relevance score, for example based upon the number of occurrences for each word. The score for each distinct word, in some implementations, is used to present related keywords for review by the user. In a particular example, the score for each distinct word may be used to generate a visualization plot for presentation to the user.

In some implementations, user adjustments are received (214). The user, for example, may be presented with the opportunity to override the automated suggestions and specify domain terms based on his/her own resources. The system can aid in user overrides, for example, by making suggestions based on the combined information of selections already made and about co-occurrence from the search. Responsive to user adjustments, in some implementations, adjustments are applied to the domain terms (216). For example, based upon addition and/or removal of domain terms by the user, a final list of domain terms may be obtained.

In some implementations, an order request is received (218). For example, a user may be presented with a user interface control for building a product order based upon the identified domain terms.

In some implementations, order components related to the domain terms are selected (220). In a microRNA-related example, the MirBase database of microRNA may be accessed to look up the identified domain terms. In some implementations, a local copy of the MirBase database may be maintained. The database, for example, lists all the known species of mature and precursor microRNA oligonucleotides. For each of the identified domain terms, optionally including a specific organism (e.g., as selected by the user), the database may be queried for matching mature microRNA species. The mature microRNA species may be used in building the order. In some implementations, the microRNA lookup may be performed prior to presenting search results to the user (in step 212), for example to provide a detailed review of information related to the search results.

In some implementations, the order is presented to the user (222). The order includes a number of components identified as being relevant to the domain terms. The order, for example, may include all of the microRNA species retrieved in step 220. Additionally, the user may include other targets, including internal or external controls, or other microRNA species not identified via the domain terms. As an example, additional microRNAs that are not included in the results may be suggested (e.g., during order presentation), based on co-occurrence in the literature; alternatively, the suggestion may be based on the user's past purchases.

After presentation of the order, in some implementations, the user may complete the order request. A custom product, in some implementations, is built for the user, based upon the identified product components. The custom product, in one example, may include all reagents necessary to detect all target organisms selected by the user. In another example, in the field of biomolecule detection, the custom product may include custom panels for the detection of proteins, messenger RNAs, single-nucleotide polymorphisms, or genetic variations.

Variations on the method 200 are possible. For example, in some implementations, the method 200 may be used to analyze multiple domains in concert, such as microRNA and proteins. The co-occurrence of microRNA and proteins in the search results, for example, would link them and contribute additional expertise to the product selection. This information could also be used for cross-recommendations. For example, customers building a protein panel could receive a recommendation for a panel that contains microRNA regulating the expression of those proteins.

The domain terms identified by step 208, with or without user modifications, in some implementations, can be used to look up pre-designed kits, such as biological panels, in the product database. The kits, for example, can be recommended to the user as an alternative to a fully customized product. Kit identification, for example, could provide customers with an easier way to finish the order process, and the vendor an easier way to deliver the product.

In some implementations, statistical information regarding the method 200 may be collected and logged for future use. For example, the information can be used by the vendor to design new pre-designed kits (e.g., panels) that correspond to common customer needs. In another example, a vendor may pre-order components based on an anticipated need as inferred from past customer behavior.

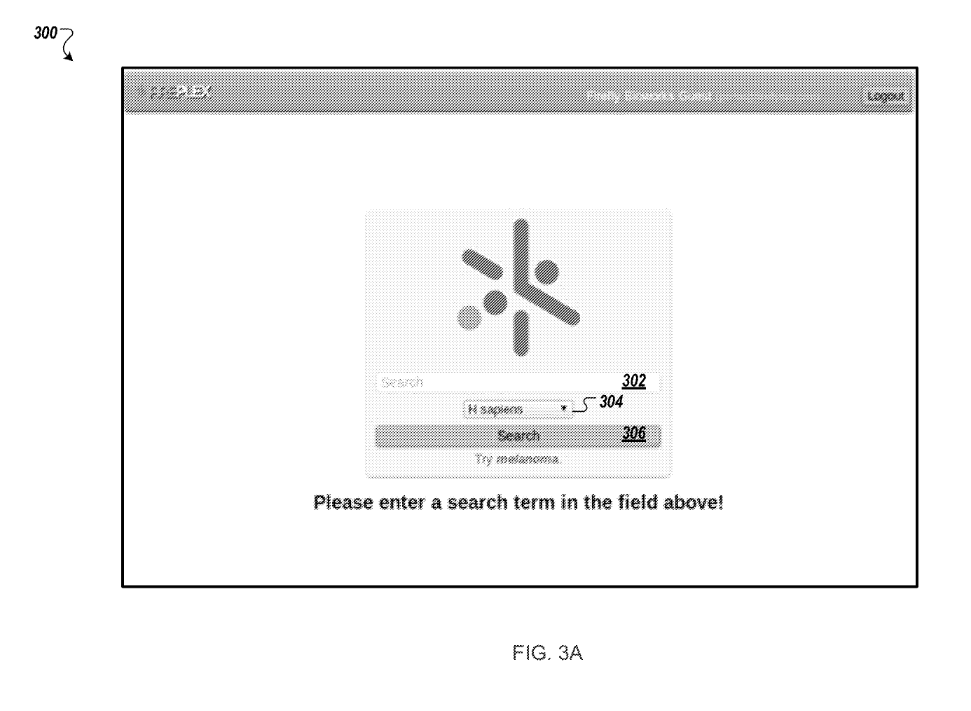

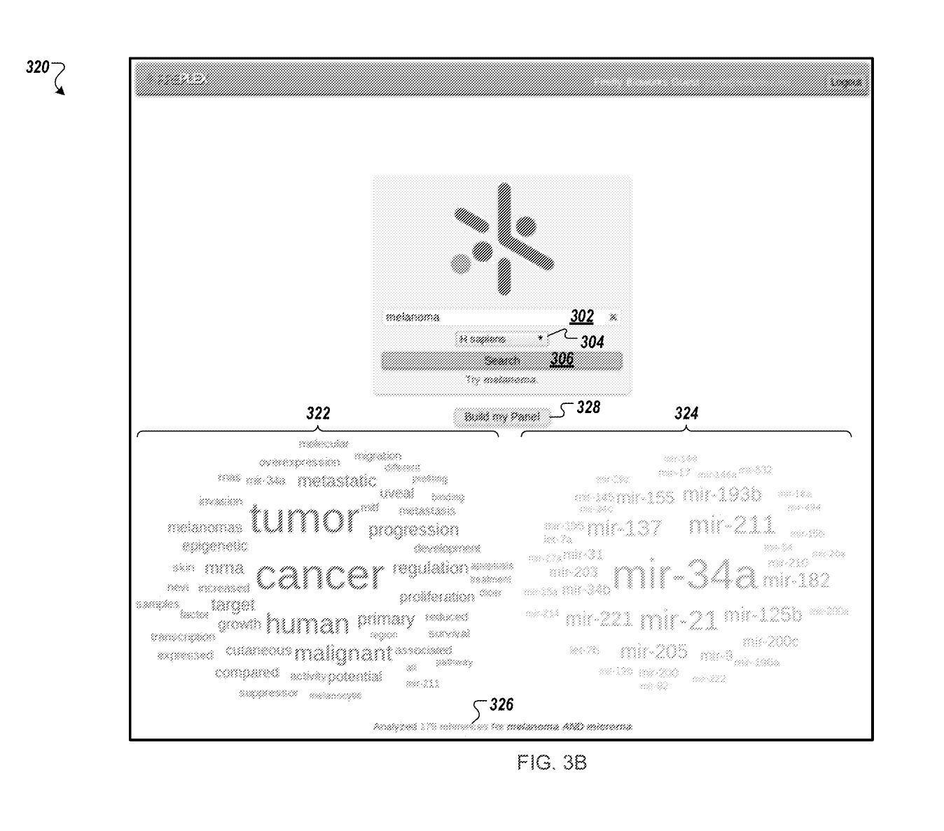

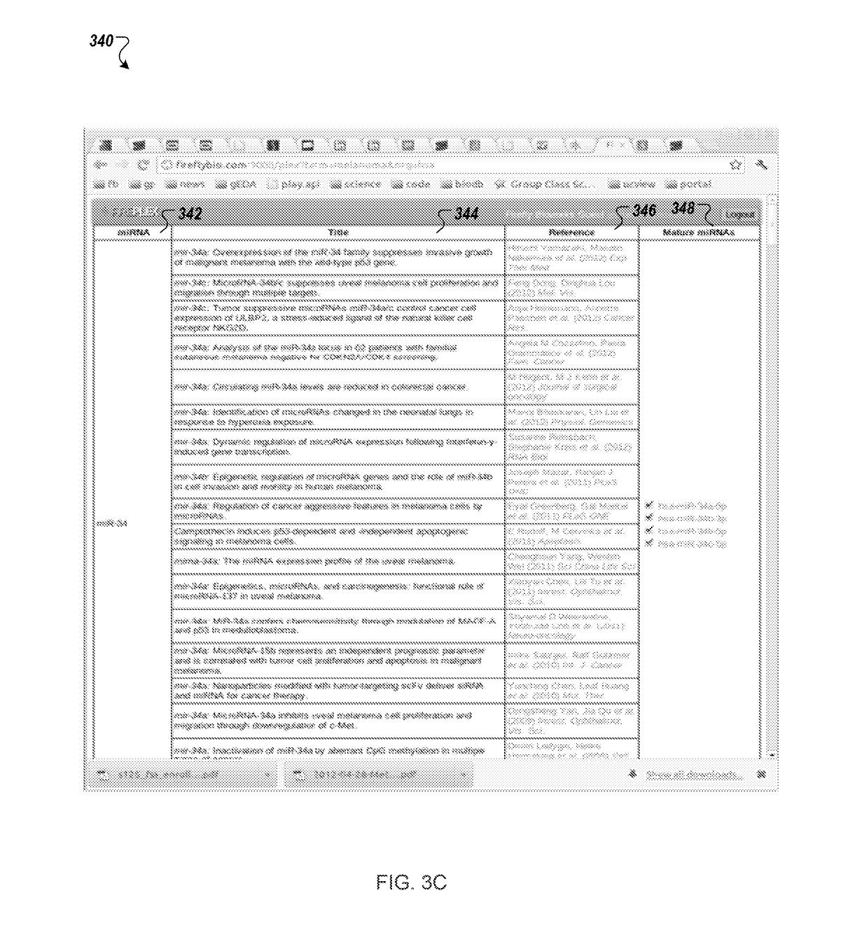

FIGS. 3A through 3C are example screen shots of a user interface for ordering a product that has been automatically customized based upon literature search results. The screen shots, for example, may be provided by the server 108 for presentation in the graphical interface 106. The user interface may be accessed, in some implementations, via a user login (e.g., user name and password). Upon login, for example, the system may compare the provided credentials with a database and, if they are identified, display a search screen 300, as illustrated in FIG. 3A. Optionally, the login screen can be bypassed to provide a guest account or access control can be left out completely.

Turning to FIG. 3A, the search screen 300 includes a search field 302, a target organism drop-down menu 304, and a search control 306. The search field 302, for example, may accept one or more terms (e.g., words, phrases, Boolean operators, etc.). The drop-down menu, as illustrated, identifies a target organism (e.g., human, mammal, etc.). In other implementations, the drop-down menu may be used to identify a domain (e.g., microRNA, proteins, etc.). The search control 306, upon selection, may submit the terms entered within the search field 302, along with the target organism identified within the target organism drop-down menu 304, as input terms for a literature search.

Turning to FIG. 3B, an initial results screen 320 contains all the elements of the Search screen 300 (as illustrated in FIG. 3A) in order to allow the user to perform another search if needed. In some embodiments, the initial results screen 320 contains two cloud representations 322, 324 of the current search result. In other implementations, the results may also be displayed as directed graphs, bar graphs, Venn Diagrams, heat maps, etc. The cloud representations (cloud maps) are designed to convey an immediate visualization of the frequency with which particular terms occur in the search result, i.e. within the titles and abstracts of the retrieved publications. The first cloud representation 322, as illustrated, lists any words found in the text, excluding the most commonly used English words. The font size, for example, indicates the frequency of the word. The second cloud representation 324 similarly presents all occurrences of terms that have been recognized by the system as microRNA designations.

There are multiple ways the user can proceed from the initial results screen 320. For example, the user may type in another search term to the search field 302 to create a new set of search results. In another example, the user may select a selectable message 326 at the bottom of the page to proceed to a detailed results screen 340. In a further example, the user may select a particular term in one of the cloud representations 322, 324. For example, the user may select one of the microRNA names in the second cloud representation 324 to proceed to a corresponding section of the detailed results screen 340. In another example, the user may select a “Build my Panel” control 328 to prepare an order form prefilled with the microRNA species listed in the second cloud representation 324. In some embodiments, words from at least one of the first cloud representation 322 and the second cloud representation 324 may be “dragged” into one or more regions (not illustrated). For example, terms may be dragged into regions designated for building (adding to) a customized product, further inspection (detailed review), and/or removal (deletion from the customized product).

In some embodiments, additional contextual information may also be displayed in the result plots. For example, particular microRNAs in the second cloud representation 324 may be colored uniquely to indicate if that target was up-regulated or down-regulated in the context related to the user's search. In another example, a subset of the microRNAs in the second cloud representation 324 may be assigned similar colors if they belong to the same “cluster” in the genome, act on the same gene, or were published in the same work. Other contextual information that may be visually represented in some manner within one or both of the cloud representations 322, 324 may include biological pathways, target abundance, species, tissues, physiological states, etc.

Turning to FIG. 3C, the detailed results screen 340 lists the search results in detail. For each microRNA designation found in the search (e.g., in a first column 342), a list of publications is given with title (e.g., in a second column 344) and first and last author, year and journal (e.g., in a third column 346). In a fourth column 348, a list of the specific mature microRNA species that are available for the designation in the first column 342 is given. This list is filtered to contain only microRNAs found in the target organism selected previously on the search screen 300 as illustrated in FIG. 3A (e.g., homo sapiens via the menu 304). On the detailed results screen 340, the user can proceed, for example, by selecting one of the literature references to be sent to an external site (such as PubMed) providing details on the referenced article. In another example, the user may select a particular mature microRNA identifier in the fourth column 348 to be sent to an external site (such as MirBase) providing details on the referenced microRNA species. In some embodiments, instead of a direct link to an external site, the system could display detailed information on each publication or entity, for example with details about the context in which they were found. In some implementations, the publication link could lead to a page that displays the title, abstract and other information about the publication, with the identified terms highlighted in colors corresponding to their domain. In some embodiments, links from a domain term may lead to a page where detailed information on the referenced entity is displayed, including a summary of relationships to other entities with which it is mentioned in the same paragraph or sentence. In some embodiments, microRNAs listed in the detailed results screen 340 can be included or excluded from the product build.

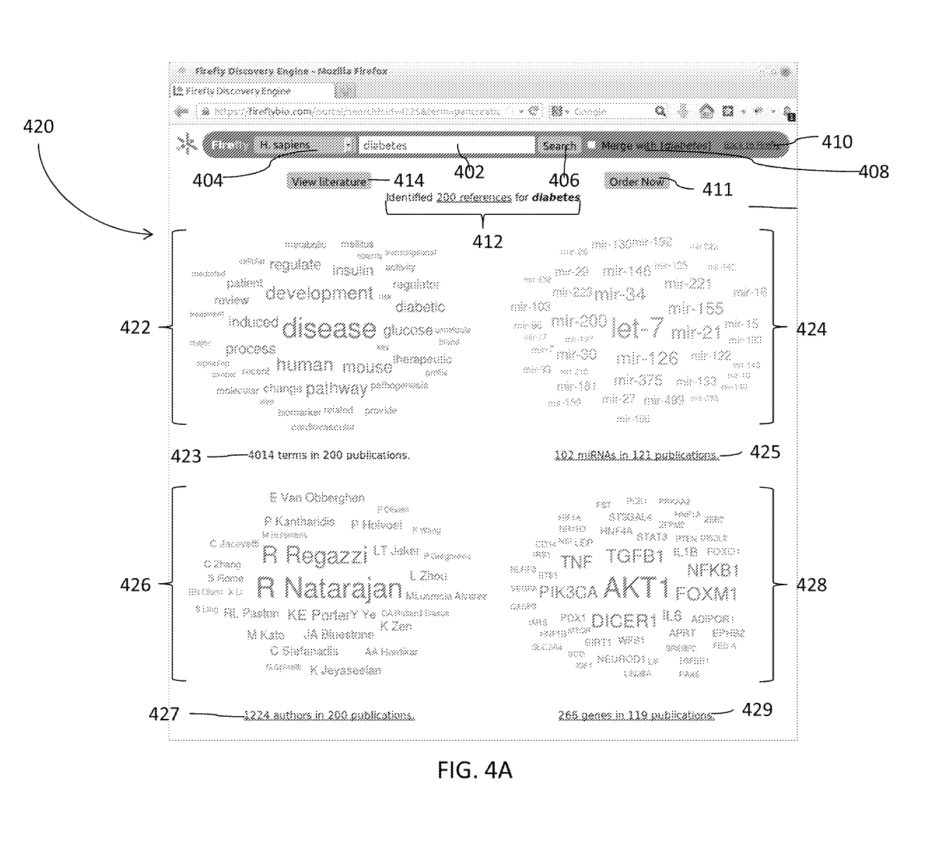

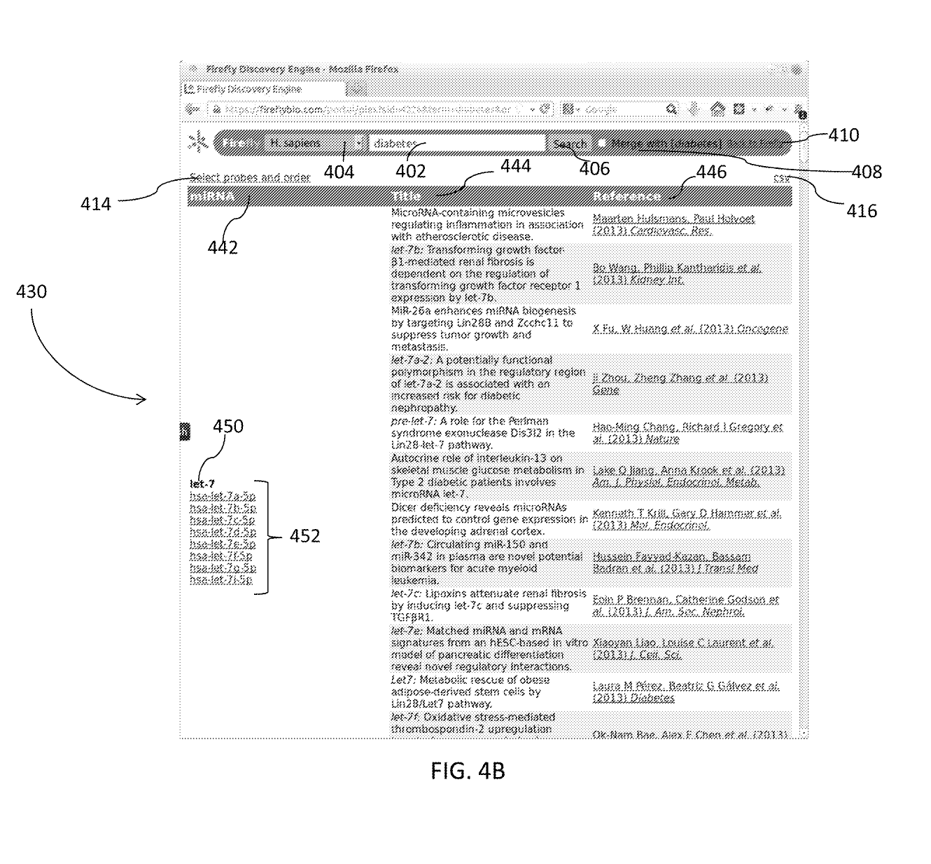

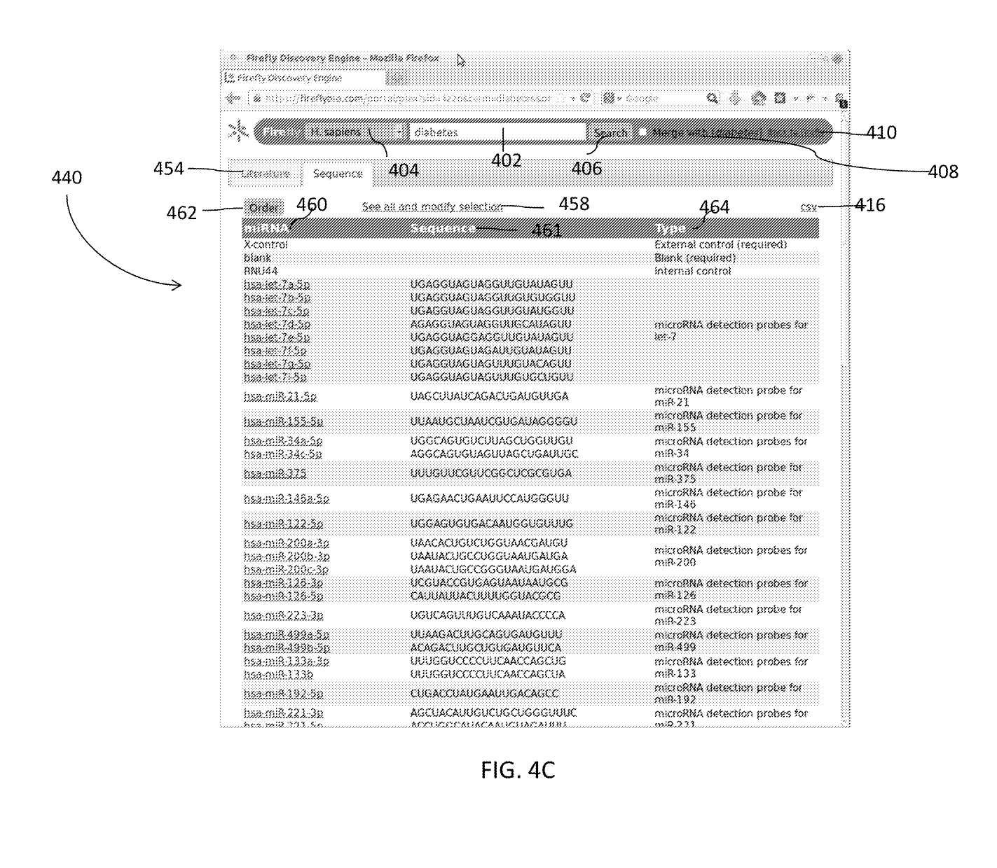

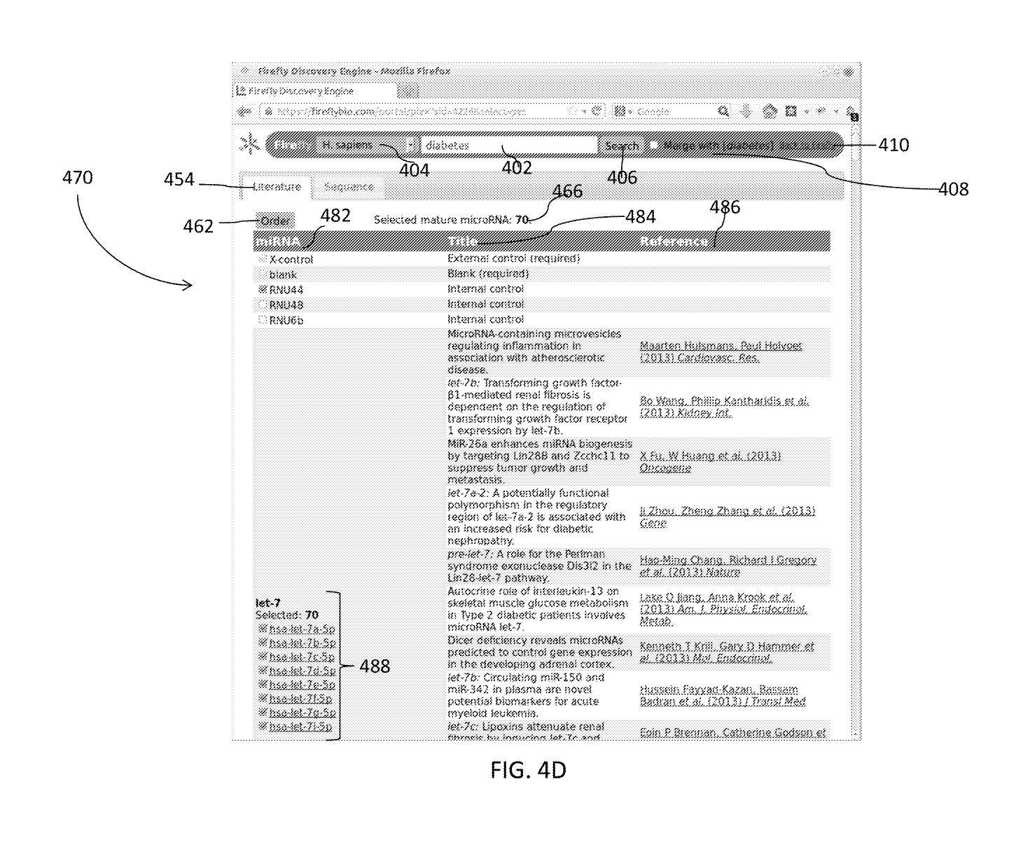

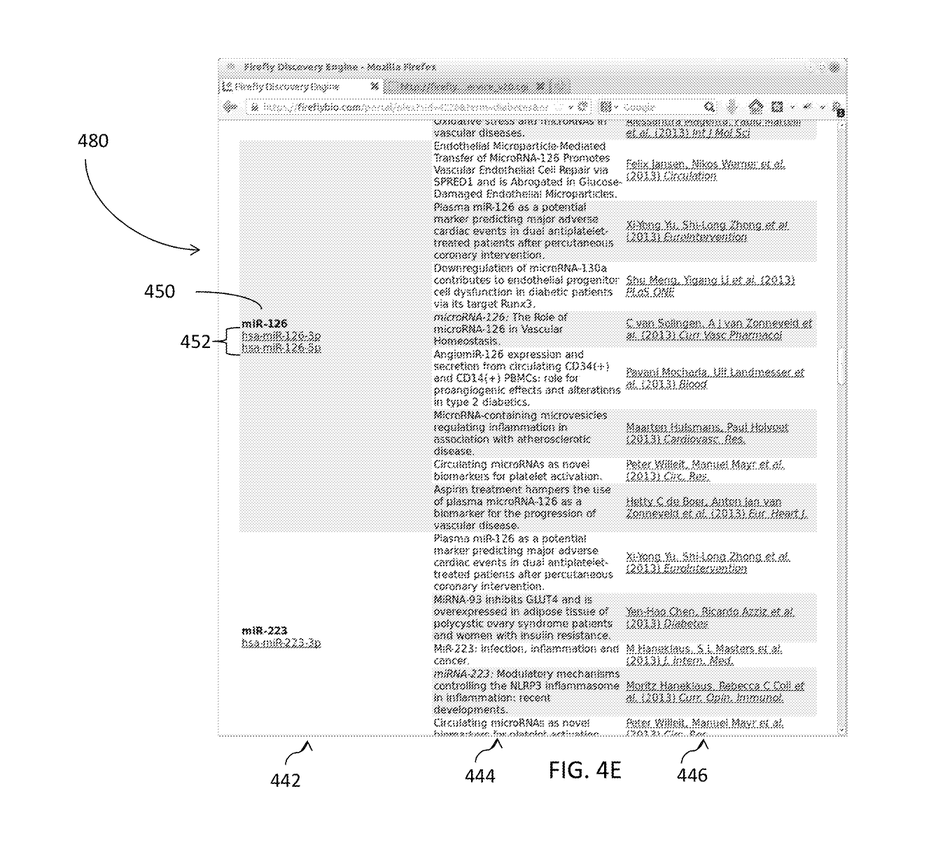

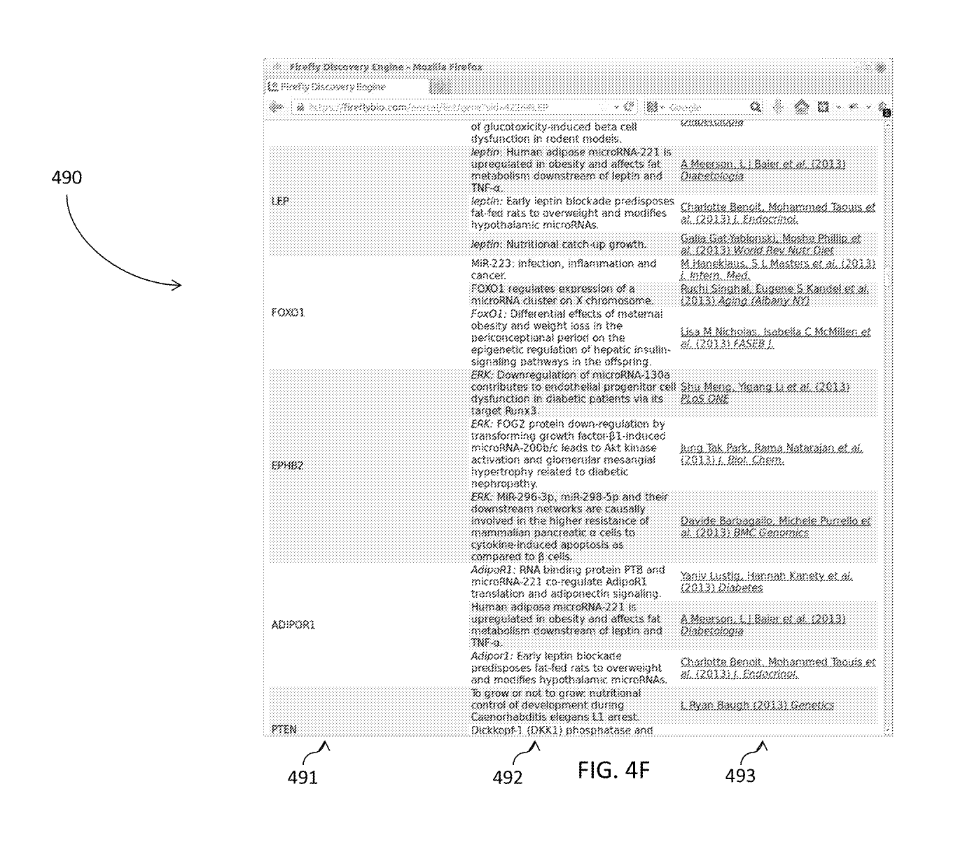

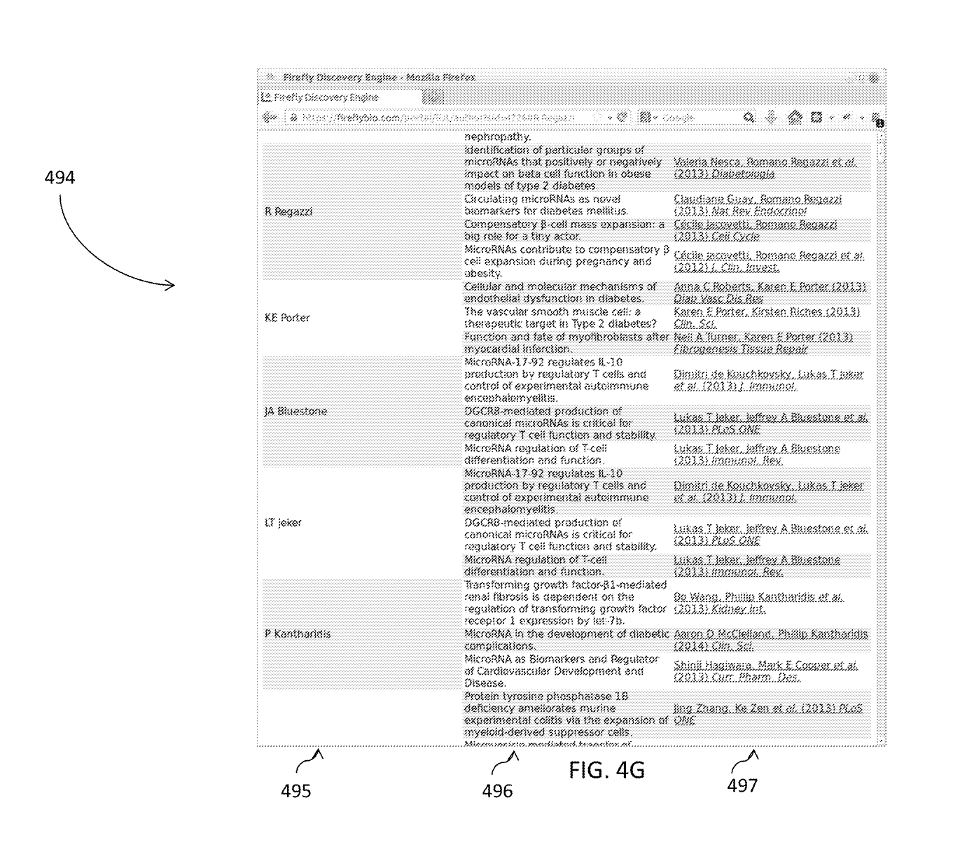

FIGS. 4A through 4G are example screen shots of a user interface for ordering a product that has been automatically customized based upon literature search results. The screen shots, for example, may be provided by the server 108 for presentation in the graphical interface 106. The user interface may be accessed, in some implementations, via a user login (e.g., user name and password). Upon login, for example, the system may compare the provided credentials with a database and, if they are identified, display a search screen 300, as illustrated in FIG. 3A. Optionally, the login screen can be bypassed to provide a guest account or access control can be left out completely.

Turning to FIG. 4A, an initial results screen 420 may contains all the elements of the search screen 300 (as illustrated in FIG. 3A) in order to allow the user to perform another search if needed. The initial results screen 420 includes a search field 402, a target organism drop-down menu 404, and a search control 406. The search field 402, for example, may accept one or more terms (e.g., words, phrases, Boolean operators, etc.). The drop-down menu, as illustrated, identifies a target organism (e.g., human, mammal, etc.). In other implementations, the drop-down menu may be used to identify a domain (e.g., microRNA, proteins, etc.). The search control 406, upon selection, may submit the terms entered within the search field 402, along with the target identified within the target organism drop-down menu 404, as input terms for a literature search.

The initial results screen 420 may further include a merge option control 408 (shown as “Merge with [diabetes] in FIG. 4A). In one example, when a user selects the merge option control 408, the results of the next search (using, e.g., a different search term, search terms and/or target organisms) are merged with the search results currently displayed (via the initial results screen 420) to analyze a larger number of publications. The initial results screen 420 may also include a back to website link 410. In one example, a user may click the back to website link to proceed back to the search section of the company website (e.g., search screen 300 as illustrated in FIG. 3A).

The initial results screen 420 may also include an order now control 411. In one example, a user may click the order now control 411 to proceed to a finished order, which is created based on current selections (which may be modified further) and presented to the user via Order screen 440 (shown in FIG. 4C). The initial results screen 420 may also include a total number of results link 412. The link 412 indicates how many references (i.e., total number of references) were identified for the particular search term (e.g., in FIG. 4A 200 references were identified for search term “diabetes”). In one example, the user may click the link 412 to proceed to another screen listing all the located references (sorted in, e.g., chronological order, last name order, title of the reference order, or any other suitable order).

In some embodiments, the initial results screen 420 may contain four cloud representations 422, 424, 426, and 428 of the current search result. In other implementations, the results may also be displayed as directed graphs, bar graphs, Venn Diagrams, heat maps, or any other suitable representation. The cloud representations (cloud maps) are designed to convey an immediate visualization of the frequency with which particular terms occur in the search result, i.e. within the titles and abstracts of the retrieved publications. The first cloud representation 422, as illustrated, lists any words found in the text, excluding the most commonly used English words. The font size and the location in the cloud, for example, indicate the frequency of the word. In some embodiments, the words located in the center of the cloud are the most frequently encountered words. The second cloud representation 424 similarly presents all occurrences of terms that have been recognized by the system as microRNA designations. The third cloud representation 426, as illustrated, lists all authors of the publications that have been recognized by the system as associated with the current search term or terms (e.g., “diabetes”). The fourth cloud representation 428, as illustrated, lists all occurrences of terms that have been recognized by the system as gene designations associated with the current search term or terms (e.g., “diabetes”). The number of terms found in each cloud representation may be customizable.

In some embodiments, directly underneath each cloud 422, 424, 426, and 428, a counter 423, 425, 427, and 429, respectively, is presented. The first counter 423 is associated with the first cloud 422. The first counter 423 presents a total number of terms associated with the given search term(s) (e.g., “diabetes”) and presents a total number of publications that these terms were found in (e.g., 200 publications for the search term “diabetes”). In FIG. 4A, the first counter 423 indicates that 4014 terms were located in 200 publications. Similarly, the second counter 425 is associated with the second cloud 424. The second counter 425 presents a total number of occurrences of terms that have been recognized by the system as microRNA designations and presents a total number of publications that these terms were found in. The third counter 427 is associated with the third cloud 426. The third counter 427 presents a total number of authors that have been identified in the search and presents a total number of publications that these authors are listed in. The fourth counter 429 is associated with the fourth cloud 428. The fourth counter 429 presents a total number of genes identified in the total number of publications located for a particular search term (e.g., “diabetes”).

In some embodiments, the counters 425, 427, and 429 are links. A user may click the link 425 to proceed to a search screen listing the different miRNAs and the publications associated with those miRNAs. Similarly, a user may click the links 427 or 429 to proceed to a search screen listing the different authors or genes, respectively, and the publications associated with those authors or genes.

In some embodiments, the counters 423, 425, 427, and 429 may be presented directly beneath their respective clouds 422, 424, 426, and 428, In other embodiments, the counters 423, 425, 427, and 429 may be located in any other suitable location within the initial search results screen 420. In some embodiments, the counters 423, 425, 427, and 429 may be presented on a separate search screen.

There are multiple ways a user can proceed from the initial results screen 420. For example, the user may type in another search term to the search field 402 to create a new set of search results, with or without clicking the merge option control 408. In another example, the user may click the link 410 to go back to the company website (and e.g., initiate a new search). In another example, the user may click links 423, 425 or 427. In another example, the user may click the view literature control 414 to proceed to a view literature screen 430 discussed below in reference to FIG. 4B. In another example, the user may click the order now link 411 to proceed to an order now screen 440 discussed below in reference to FIG. 4C.

In another example, the user may select (and click on) a particular search term in the clouds 422, 424, 426, or 428 to proceed to a search screen focused on the selected search term. For example, the user may select one of the microRNA names (e.g., Mir 126) in the second cloud representation 424 to proceed to a corresponding section of the detailed results screen 480 as discussed below in FIG. 4E.

Turning now to FIG. 4B, the view literature screen 430 illustrates a detailed listing of all referenced microRNAs in order of score (based on, e.g., the number of times each particular microRNA is mentioned in the literature) with all the references where these microRNAs are mentioned. In some embodiments, the score is based on frequency of occurrence and number of sequencing reads that substantiate the mature species of microRNA in mirBase. In some embodiments, the view literature screen 430 includes a csv link 416. The user may click the csv link 416 to download the displayed data as character-separated values (csv). In some embodiments, the view literature screen 430 includes a “select probes and order” link 418. In some embodiments, the user may click the “select probes and order” link 418 to proceed to a probe selection view, where the user may select and customize the different probes for the order,

In some embodiments, the view literature screen 430 may include a miRNA column 442, a title column 444, and a reference column 446. The miRNA column 442 may include generic names 450 for the microRNA (e.g., associated with the search term(s)) and a list of links 452 directly underneath each generic names 450 to specific mature species that lead to the mirBase entry for that species. The title column 444 may include a listing of titles of each publication in which a reference to the corresponding microRNA (listed in miRNA column 442) was found. The reference column 446 may include a listing of the bibliographic references, with links to those references in PubMed.

Turning now to FIG. 4C, the order now screen 440 may illustrate a listing of the suggested microRNAs that should be ordered for a complete panel. This listing may be ordered as illustrated in FIG. 4C (e.g., with no further decisions or changes to be made by the user) or the listing may optionally be modified at the selection of the user.

In some embodiments, the order now screen 440 includes a literature tab 454. The user may select the literature tab 454 to proceed to the view literature screen 430 discussed in reference to FIG. 4B. In some embodiments, the order now screen 440 also includes a “see all and modify selection” link 458. In one example, the user may click the link 458 to proceed to a panel selection screen 470 discussed in reference to FIG. 4D. In some embodiments, the order now screen 440 also includes an order tab 462. The user may click the order tab 462, which causes the complete panel to be transferred to a commercial ordering system.

In some embodiments, the order now screen 440 includes three columns: miRNA column 460, sequence column 461, and type column 464. The miRNA column 460 may include a listing of target(s) and/or control(s) to be included in the panel. Each target in column 460 may be provided as a link (that may be clicked by the user) to the mirBase entry associated with each target. The sequence column 461 provides a listing of respective sequences associated with each target in the miRNA column 460. The type column 464 provides a listing of further descriptions of each target and control listed in the miRNA column 460.

Turning now to FIG. 4D, the panel selection screen 470 presents the user with options to pick and choose the targets identified in the search results. In some embodiments, the panel selection screen 470 includes a counter of the selected mature microRNA targets 466, which sums up the total number of currently selected targets (to be included in the order). In some embodiments, the counter 466 may turn a different color (e.g., red) and may e.g., include a warning if the multiplex limit is exceeded. In some embodiments, the panel selection screen 470 may include three columns: miRNA column 482, title column 484, and reference column 486. In some embodiments, the miRNA column 482 provides a listing of controls and targets that are available for selection, Some controls are required (e.g., the X-control and the blank control). Some controls are optional and may be selected by the user. If a checkbox adjacent to a target is checked, that target is selected for the order. If a checkbox adjacent to a target is not checked, that target is not selected for the order. In some embodiments, the title column 484 includes a listing of titles of each publication in which a reference to the corresponding microRNA (listed in miRNA column 482) was found. In some embodiments, the reference column 486 includes a listing of the bibliographic references, with links to those references in PubMed.

As discussed above, in some examples, a user may select a desired miRNA in the second cloud 424 of FIG. 4A. A user may, for example, select “Mir 126” in the second cloud 424 to proceed to a Mir 126-focused results screen 480 illustrated in FIG. 4E. The Mir 126 results screen 480 includes three columns: the miRNA column 442, the title column 444, and the reference column 446. The three columns 442, 444, and 446 may be identical to the ones shown in FIG. 4B, except that the screen is scrolled down to the location in the miRNA column 442 where the generic name (e.g., miR-126 in FIG. 4D) is found. Underneath the generic name 450, a list of links to specific mature species that lead to the mirBase entry for that species (e.g., miR-126) is provided.

As discussed above, in some examples, a user may select a desired gene in the fourth cloud 428 of FIG. 4A. A user may, for example, select “LEP” (leptin) in the fourth cloud 428 to proceed to a LEP-focused results screen 490 illustrated in FIG. 4F. The LEP results screen 490 may include three columns: a gene column 491, a title column 492, and a reference column 493. The columns 492 and 493 may be similar to the ones shown in FIG. 4B, except that the screen is scrolled down to the location in the gene column 491 where LEP is found. The title column 492 may provide a list of the titles associated with each gene listed in the gene column 491. In some embodiments, the reference column 493 includes a listing of the bibliographic references associated with each respective title in the title column 492, with links to those references in PubMed.

As discussed above, in some examples, a user may select a desired author in the third cloud 426 of FIG. 4A. A user may, for example, select author “R Regazzi” in the third cloud 426 to proceed to a R Regazzi-focused results screen 494 illustrated in FIG. 4G. In some embodiments, the R Regazzi results screen 494 includes three columns: an author column 495, a title column 496, and a reference column 497. In some embodiments, the columns 496 and 497 may be similar to the ones shown in FIG. 4B, except that the screen is scrolled down to the location in the author column 495 where R Regazzi is found. The title column 496 may provide a list of the titles associated with each author listed in the author column 495. In some embodiments, the reference column 497 may include a listing of the bibliographic references associated with each respective title in the title column 496, with links to those references in PubMed.

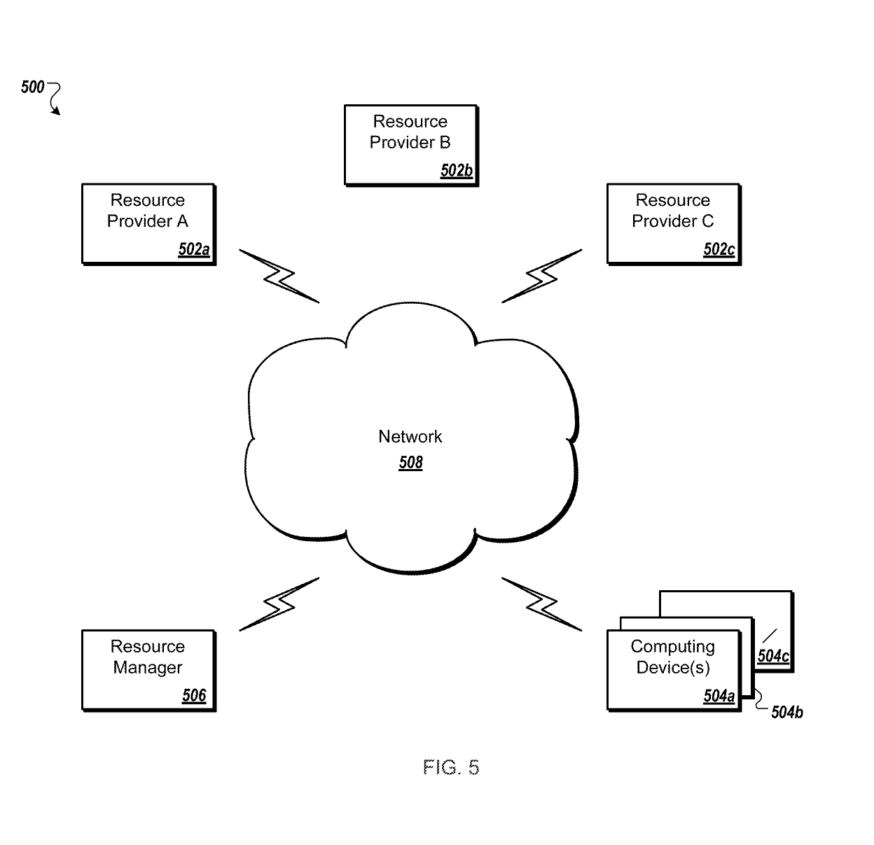

As shown in FIG. 5, an implementation of an exemplary cloud computing environment 500 for automated product customization based on literature search results is shown and described. The cloud computing environment 500 may include one or more resource providers 502a, 502b, 502c (collectively, 502). Each resource provider 502 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 502 may be connected to any other resource provider 502 in the cloud computing environment 500. In some implementations, the resource providers 502 may be connected over a computer network 508. Each resource provider 502 may be connected to one or more computing device 504a, 504b, 504c (collectively, 504), over the computer network 508.

The cloud computing environment 500 may include a resource manager 506. The resource manager 506 may be connected to the resource providers 502 and the computing devices 504 over the computer network 508. In some implementations, the resource manager 506 may facilitate the provision of computing resources by one or more resource providers 502 to one or more computing devices 504. The resource manager 506 may receive a request for a computing resource from a particular computing device 504. The resource manager 506 may identify one or more resource providers 502 capable of providing the computing resource requested by the computing device 504. The resource manager 506 may select a resource provider 502 to provide the computing resource. The resource manager 506 may facilitate a connection between the resource provider 502 and a particular computing device 504. In some implementations, the resource manager 506 may establish a connection between a particular resource provider 502 and a particular computing device 504. In some implementations, the resource manager 506 may redirect a particular computing device 504 to a particular resource provider 502 with the requested computing resource.

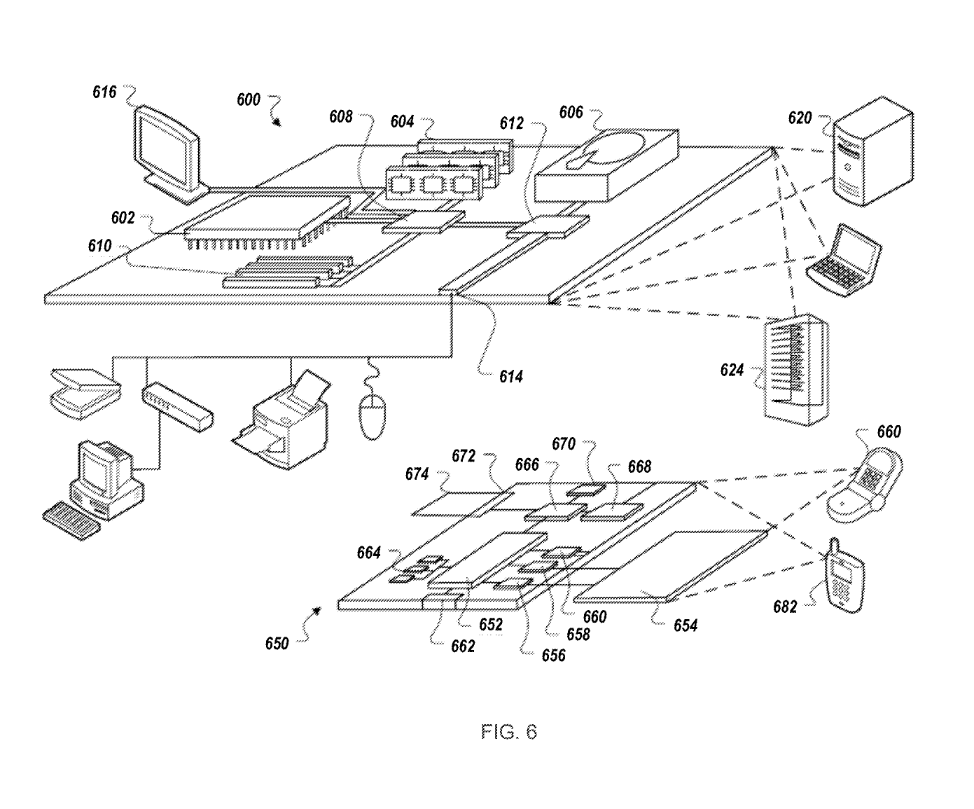

FIG. 6 shows an example of a computing device 600 and a mobile computing device 650 that can be used to implement the techniques described in this disclosure. The computing device 600 is intended to represent various forms of digital computers, such as laptops, desktops, workstations, personal digital assistants, servers, blade servers, mainframes, and other appropriate computers. The mobile computing device 650 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 600 includes a processor 602, a memory 604, a storage device 606, a high-speed interface 608 connecting to the memory 604 and multiple high-speed expansion ports 610, and a low-speed interface 612 connecting to a low-speed expansion port 614 and the storage device 606. Each of the processor 602, the memory 604, the storage device 606, the high-speed interface 608, the high-speed expansion ports 610, and the low-speed interface 612, are interconnected using various busses, and may be mounted on a common motherboard or in other manners as appropriate. The processor 602 can process instructions for execution within the computing device 600, including instructions stored in the memory 604 or on the storage device 606 to display graphical information for a GUI on an external input/output device, such as a display 616 coupled to the high-speed interface 608. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system).

The memory 604 stores information within the computing device 600. In some implementations, the memory 604 is a volatile memory unit or units. In some implementations, the memory 604 is a non-volatile memory unit or units. The memory 604 may also be another form of computer-readable medium, such as a magnetic or optical disk.

The storage device 606 is capable of providing mass storage for the computing device 600. In some implementations, the storage device 606 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 602), perform one or more methods, such as those described above. The instructions can also be stored by one or more storage devices such as computer- or machine-readable mediums (for example, the memory 604, the storage device 606, or memory on the processor 602).

The high-speed interface 608 manages bandwidth-intensive operations for the computing device 600, while the low-speed interface 612 manages lower bandwidth-intensive operations. Such allocation of functions is an example only. In some implementations, the high-speed interface 608 is coupled to the memory 604, the display 616 (e.g., through a graphics processor or accelerator), and to the high-speed expansion ports 610, which may accept various expansion cards (not shown). In the implementation, the low-speed interface 612 is coupled to the storage device 606 and the low-speed expansion port 614. The low-speed expansion port 614, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 600 may be implemented in a number of different forms, as shown in the figure. For example, it may be implemented as a standard server 620, or multiple times in a group of such servers. In addition, it may be implemented in a personal computer such as a laptop computer 622. It may also be implemented as part of a rack server system 624. Alternatively, components from the computing device 600 may be combined with other components in a mobile device (not shown), such as a mobile computing device 650. Each of such devices may contain one or more of the computing device 600 and the mobile computing device 650, and an entire system may be made up of multiple computing devices communicating with each other.

The mobile computing device 650 includes a processor 652, a memory 664, an input/output device such as a display 654, a communication interface 666, and a transceiver 668, among other components. The mobile computing device 650 may also be provided with a storage device, such as a micro-drive or other device, to provide additional storage. Each of the processor 652, the memory 664, the display 654, the communication interface 666, and the transceiver 668, are interconnected using various buses, and several of the components may be mounted on a common motherboard or in other manners as appropriate.

The processor 652 can execute instructions within the mobile computing device 650, including instructions stored in the memory 664. The processor 652 may be implemented as a chipset of chips that include separate and multiple analog and digital processors. The processor 652 may provide, for example, for coordination of the other components of the mobile computing device 650, such as control of user interfaces, applications run by the mobile computing device 650, and wireless communication by the mobile computing device 650.

The processor 652 may communicate with a user through a control interface 658 and a display interface 656 coupled to the display 654. The display 654 may be, for example, a TFT (Thin-Film-Transistor Liquid Crystal Display) display or an OLED (Organic Light Emitting Diode) display, or other appropriate display technology. The display interface 656 may comprise appropriate circuitry for driving the display 654 to present graphical and other information to a user. The control interface 658 may receive commands from a user and convert them for submission to the processor 652. In addition, an external interface 662 may provide communication with the processor 652, so as to enable near area communication of the mobile computing device 650 with other devices. The external interface 662 may provide, for example, for wired communication in some implementations, or for wireless communication in other implementations, and multiple interfaces may also be used.

The memory 664 stores information within the mobile computing device 650. The memory 664 can be implemented as one or more of a computer-readable medium or media, a volatile memory unit or units, or a non-volatile memory unit or units. An expansion memory 674 may also be provided and connected to the mobile computing device 650 through an expansion interface 672, which may include, for example, a SIMM (Single In Line Memory Module) card interface. The expansion memory 674 may provide extra storage space for the mobile computing device 650, or may also store applications or other information for the mobile computing device 650. Specifically, the expansion memory 674 may include instructions to carry out or supplement the processes described above, and may include secure information also. Thus, for example, the expansion memory 674 may be provide as a security module for the mobile computing device 650, and may be programmed with instructions that permit secure use of the mobile computing device 650. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. that the instructions, when executed by one or more processing devices (for example, processor 652), perform one or more methods, such as those described above. The instructions can also be stored by one or more storage devices, such as one or more computer- or machine-readable mediums (for example, the memory 664, the expansion memory 674, or memory on the processor 652). In some implementations, the instructions can be received in a propagated signal, for example, over the transceiver 668 or the external interface 662.

The mobile computing device 650 may communicate wirelessly through the communication interface 666, which may include digital signal processing circuitry where necessary. The communication interface 666 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 668 using a radio-frequency. In addition, short-range communication may occur, such as using a Bluetooth®, Wi-Fi™, or other such transceiver (not shown). In addition, a GPS (Global Positioning System) receiver module 670 may provide additional navigation- and location-related wireless data to the mobile computing device 650, which may be used as appropriate by applications running on the mobile computing device 650.

The mobile computing device 650 may also communicate audibly using an audio codec 660, which may receive spoken information from a user and convert it to usable digital information. The audio codec 660 may likewise generate audible sound for a user, such as through a speaker, e.g., in a handset of the mobile computing device 650. Such sound may include sound from voice telephone calls, may include recorded sound (e.g., voice messages, music files, etc.) and may also include sound generated by applications operating on the mobile computing device 650.

The mobile computing device 650 may be implemented in a number of different forms, as shown in the figure. For example, it may be implemented as a cellular telephone 680. It may also be implemented as part of a smart-phone 682, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In view of the structure, functions and apparatus of the systems and methods described here, in some implementations, environments and methods for automated product customization based on literature search results are provided. Having described certain implementations of methods and apparatus for supporting automated product customization based on literature search results, it will now become apparent to one of skill in the art that other implementations incorporating the concepts of the disclosure may be used. Therefore, the disclosure should not be limited to certain implementations, but rather should be limited only by the spirit and scope of the following claims.

1. A method comprising:
receiving, via a network, one or more user search terms entered by a user at a remote computing device;
constructing, by a processor of a computer device, a query, wherein the query comprises the one or more user search terms and/or is determined based at least in part on the one or more user search terms;
obtaining, from one or more third party literature repositories, a plurality of search results responsive to the query;
identifying, by the processor, one or more scientific domain terms within the plurality of search results, wherein each of the one or more scientific domain terms relates to one or more of a set of predetermined scientific domain categories;
prioritizing, by the processor, the one or more scientific domain terms based at least in part upon frequency of occurrence within the plurality of search results;
selecting, by the processor, one or more order components relating to a tangible product, wherein each order component of the one or more order components relates to a respective scientific domain term of the one or more scientific domain terms; and
providing, for display to the user at the remote computing device, order information comprising the one or more order components.

2. The method of claim 1, wherein the set of predetermined scientific domain categories comprises one or more of molecular biology, proteins, immunoassays, genetic polymorphisms, genomic assays, RNA molecules, expression analysis, messenger RNA, small RNA molecules, and/or microRNA molecules.

3. The method of any one of the preceding claims, wherein a first repository of the one or more third party literature repositories is PubMed.

4. The method of any one of the preceding claims, wherein the query comprises a domain term.

5. The method of claim 4, wherein constructing the query comprises identifying the domain term relevant to the user.

6. The method of claim 5, wherein identifying the domain term comprises identifying the scientific domain based upon the one or more terms entered by the user.

7. The method of any one of the preceding claims, wherein identifying the one or more domain terms comprises using a pattern matching algorithm to identify patterns within text of the plurality of search results.

8. The method of claim 7, wherein each search result of the plurality of search results comprises at least one of a summary, an abstract, an article, and a publication.

9. The method of any one of the preceding claims, further comprising filtering the plurality of search results to remove duplicates.

10. The method of any one of the preceding claims, further comprising filtering the one or more domain terms to remove one or more least frequently identified domain terms.

11. The method of any one of the preceding claims, wherein prioritizing the one or more scientific domain terms comprises scoring the one or more scientific domain terms by at least the frequency of occurrence.

12. The method of claim 11, further comprising, prior to selecting the one or more order components, providing, for display to the user at the remote computing device, the one or more domain terms, wherein providing the one or more domain terms comprises providing a respective score associated with each domain term of the one or more domain terms.

13. The method of claim 12, further comprising receiving, via the network, responsive to providing the one or more domain terms, at least one of an addition of a domain term and a removal of a domain term of the one or more domain terms.

14. The method of claim 12, further comprising, prior to selecting the one or more order components, receiving an indication from the user to initiate generation of a product order based upon the one or more domain terms.

15. The method of any one of the preceding claims, wherein the tangible product comprises a biological panel for detection of a plurality of identified microRNA targets, a multiplex biological panel, a therapeutic or combination of therapeutics, a PCR primer set, or one or more small RNA mimics.

16. The method of claim 15, wherein the tangible product comprises a customized multiplex biological panel for detection of one or more identified proteins, messenger RNAs, SNPs, and/or genetic variations thereof.

17. The method of claim 15, wherein the tangible product comprises one or more small RNA mimics comprising microRNAs, lncRNAs, siRNAs, anti-microRNAs, piwiRNAs, and/or any combination thereof.

18. A system comprising:
a processor; and
a memory having instructions stored thereon, wherein the instructions, when executed by the processor, cause the processor to:
receive, via a network, one or more user search terms entered by a user at a remote computing device;
construct a query, wherein the query comprises the one or more user search terms and/or is determined based at least in part on the one or more user search terms;
obtain, from one or more third party literature repositories, a plurality of search results responsive to the query;
identify one or more scientific domain terms within the plurality of search results, wherein each of the one or more scientific domain terms relates to one or more of a set of predetermined scientific domain categories;
prioritize the one or more scientific domain terms based at least in part upon frequency of occurrence within the plurality of search results;
select one or more order components relating to a tangible product, wherein each order component of the one or more order components relates to a respective scientific domain term of the one or more scientific domain terms; and
provide, for display to the user at the remote computing device, order information comprising the one or more order components.

19. A non-transitory computer readable medium having instructions stored thereon, wherein the instructions, when executed, cause the processor to:
receive, via a network, one or more user search terms entered by a user at a remote computing device;
construct a query, wherein the query comprises the one or more user search terms and/or is determined based at least in part on the one or more user search terms;
obtain, from one or more third party literature repositories, a plurality of search results responsive to the query;
identify one or more scientific domain terms within the plurality of search results, wherein each of the one or more scientific domain terms relates to one or more of a set of predetermined scientific domain categories;
prioritize the one or more scientific domain terms based at least in part upon frequency of occurrence within the plurality of search results;
select one or more order components relating to a tangible product, wherein each order component of the one or more order components relates to a respective scientific domain term of the one or more scientific domain terms; and
provide, for display to the user at the remote computing device, order information comprising the one or more order components.

20. A method for automated (or semi-automated) selection or customization of a tangible product, the method comprising:
providing a first graphical user interface for display on a user computing device, said interface being configured to accept user input from the user computing device;
receiving, via a network, a first set of user input from the user computing device, said first set of user input comprising a first set of one or more tokens and, optionally, one or more of the following: (i) a selected organism, and (ii) a selected scientific domain;
accessing, by the processor, one or more databases and performing, by the processor, a first query of the one or more databases using said first set of user input to identify a set of initial search results;
transmitting, for graphical display on the user computing device, the set of initial search results for rendering on a display of the user computing device;
providing, for graphical display on the user computing device, a graphical user interface (GUI) widget that, upon selection by the user, initiates preparation of an order form providing specifications for a customized tangible product corresponding to at least a subset of the initial (or subsequent) search results;
providing, for graphical display on the user computing device, the order form, said order form comprising a listing of selectable attributes corresponding to said specifications of the tangible product.

21. The method of claim 20, comprising providing, for graphical display on the user computing device, a link corresponding to each of one or more members of the set of initial search results rendered on the display of the user computing device, said link(s) comprising an identification of a microRNA (or messenger RNA, SNP, or genetic variation) search result which, upon selection of the link by the user, presents a listing of documents from the one or more databases describing the selected microRNA (or messenger RNA, SNP, or genetic variation) and, optionally, presents links to said documents.

22. The method of claim 20 or 21, comprising receiving, via the network, a second (or subsequent) set of user input from the user computing device following display of the set of initial (or prior) search results on the display of the user computing device, said second (or subsequent) set of user input comprising a second (or subsequent) set of one or more tokens, and performing, by the processor, a second (or subsequent) query of the one or more databases using said second (or subsequent) set of user input to identify a set of subsequent search results.

23. The method of claim 22, wherein the second (or subsequent) set of user input is a selection by the user of one or more search results previously presented to the user.

24. The method of any one of claims 20 to 23, comprising providing, for graphical display on the user computing device, a GUI widget that, upon selection by the user, instructs merging, by the processor, of the first query with the second query of the one or more databases to identify the set of subsequent search results.

25. The method of any one of claims 20 to 24, comprising providing, for graphical display on the user computing device, a plurality of graphical cloud representations, each graphical cloud representation configured to convey a visualization of a score corresponding to each of the set of initial search results, wherein the plurality of graphical cloud representations comprise one or more of the following: (i) a key word cloud comprising words found in texts identified in the first query; (ii) a microRNA cloud comprising designations of microRNAs in texts identified in the first query; (iii) an author cloud comprising designations of authors of texts identified in the first query; and (iv) a gene cloud comprising designations of genes in texts identified in the first query.

26. The method of any one of claims 20 to 25, further comprising transmitting the completed order form to a manufacturer for fulfillment of the tangible product.

27. The method of claim 26, wherein the tangible product is a new, customized product.

28. The method of claim 26, wherein the tangible product is an existing product.

29. The method of any one of claims 20 to 28, wherein the tangible product comprises a biological panel for detection of a plurality of identified microRNA targets, a multiplex biological panel, a therapeutic or combination of therapeutics, a PCR primer set, and/or one or more small RNA mimics.

30. The method of claim 29, wherein the tangible product comprises a customized multiplex biological panel for detection of one or more identified proteins, messenger RNAs, SNPs, and/or genetic variations thereof.

31. The method of claim 29, wherein the tangible product comprises one or more small RNA mimics comprising microRNAs, lncRNAs, siRNAs, anti-microRNAs, piwiRNAs, and/or any combination thereof.

32. The method of any one of claims 20 to 31, wherein the first set of one or more tokens comprises an alphanumeric user search term (word, phrase), image, graphical chemical structure, or graphical biological structure, and/or any combination thereof.

33. The method of any one of claims 20 to 32, wherein the selected organism is a human or a mammal.

34. The method of any one of claims 20 to 33, wherein the selected scientific domain comprises microRNAs, proteins, genes, and/or any combination thereof.

35. The method of any one of claims 20 to 34, wherein the one or more databases comprises biological databases, medical databases and/or scientific literature databases.

36. The method of any one of claims 20 to 35, wherein the set of initial search results is rendered on the display of the user computing device as an initial search results page comprising a plurality of graphical cloud representations, each graphical cloud representation configured to convey a visualization of a score corresponding to each of the set of initial search results.

37. The method of claim 36, wherein said score is a function of frequency of occurrence within the searched database(s).

38. The method of claim 36 or 37, wherein said score is represented graphically by font size and/or color.

39. The method of any one of claims 36-38, wherein only search results exceeding a given score are graphically displayed.

40. The method of any one of claims 20-39, wherein the GUI widget is a control button.

41. The method of any one claims 20 to 40, wherein the selectable attributes comprise specific microRNA targets, specific messenger RNAs, specific SNPs, and/or specific genetic variations.

42. The method of any one of claims 20 to 40, wherein the selectable attributes comprise specific therapeutics, elements of a PCR primer set, and/or small RNA mimics.

43. The method of any one of claims 20 to 42, wherein each of said selectable attributes comprises a GUI widget displayed on the order form allowing selection or deselection of said attribute by the user, said selected attributes (following selection by the user of a subset of said displayed selectable attributes) corresponding to the specifications for the customized tangible product.

44. The method of any one of claims 20 to 43, further comprising identifying, by the processor, one or more scientific domain terms within the set of initial search results, wherein each of the one or more scientific domain terms relates to one or more of a set of predetermined scientific domain categories.

45. The method of claim 44, further comprising prioritizing, by the processor, the one or more scientific domain terms based at least in part upon frequency of occurrence within the set of initial search results.

46. The method of any one of claims 20 to 45, wherein the one or more databases is a third party literature repository.

47. The method of claim 46, wherein at least a portion of the one or more databases is downloaded and/or stored on a server associated with the processor.

48. The method of any one of claims 20 to 45, wherein the one or more databases is stored locally on a server associated with the processor.
